(19) United States
(12) Patent Application Publication
(10) Pub. No.: US 2025/0288446 A1
(43) Pub. Date: Sep. 18, 2025

(54) CORRECTION DEVICE

(71) Applicants: Jesse Jacob JUTKOWITZ, Beijing (CN); Gaoxi LI, Beijing (CN); Metabition Health management Co., Ltd., Beijing (CN)

(72) Inventor: Jesse Jacob JUTKOWITZ, Beijing (CN)

(21) Appl. No.: 19/035,760

(22) Filed: Jan. 23, 2025

(30) Foreign Application Priority Data
Mar. 12, 2024 (CN) 202410282056.4

(51) Int. Cl. A61F 5/01

(52) U.S. Cl. CPC A61F 5/0104

(57) ABSTRACT

A correction device is disclosed, which includes a housing assembly, an electromagnet, a correction assembly, a mounting assembly and a control assembly. The housing assembly defines a receiving cavity having a clearance hole. The electromagnet includes a coil having a clearance channel, and an armature passing through the clearance channel and being movable in an axial direction of the coil. The correction assembly is disposed on the housing assembly and movable along the axial direction of the coil, with an end being connected to the armature, and a further end passing through the clearance hole. The control and mounting assemblies are disposed on the housing assembly, the control assembly being electrically connected to the coil to control the coil to be energized or de-energized. The armature, upon the coil being energized, pushes the further end of the correction assembly to move in a direction extending out of the housing assembly.

### CROSS-REFERENCE TO RELATED APPLICATION

This application is based on and claims priority to Chinese Patent Application No. 2024102820564, filed on Mar. 12, 2024, the entirety of which is incorporated by reference herein.

### TECHNICAL FIELD

The present disclosure relates to the technical field of medical instruments, and in particular to a correction device.

### BACKGROUND

Research indicates that a downward posture caused by anteroinferior subluxation of a first rib can affect breathing and the heart pulse rate. Subluxation refers to the incomplete or partial dislocation of a bone within a joint. The subluxation of the first rib may cause pain at the rib site or be completely painless, yet such a condition can lead to pain in other areas of the body. This is a quite common ailment and is difficult to correct. There are many potential causes for the subluxation of the first rib, including writing, gardening, cooking actions that involve whipping, working at a desk, prolonged typing, sleeping on your side with too small a pillow, and driving in heavy traffic. Any trauma that forces the head and/or neck forward can potentially lead to the subluxation of the first rib. Symptoms may include neck pain, headaches, carpal tunnel syndrome, chest pain, trapezius pain, upper and middle back pain, elbow, wrist, hand pain, clavicle pain, shallow breathing, and decreased heart pulse rate. These symptoms may subsequently cause other conditions.

Because the first rib is located behind the clavicle, the treatment of its anteroinferior subluxation is difficult. A known treatment method includes laterally flexing the patient's head toward the same side as the rib and rotating the head away from the rib. A stabilizing hand is pressed on the other side, pressing laterally to medially,, while supporting one side of the head and the posterolateral side of the neck. This method uses the contact point of the proximal interphalangeal joint of the index finger to apply pressure on the anterior side of the upper or posterior part of the first rib, while simultaneously using the webbing of the fingers to touch the surrounding area. A thrust is applied from top to bottom, from the lateral to the medial, or from front to back, slightly downward, pushing toward the inferior angle of the scapula on the opposite side.

Currently, patients with anteroinferior subluxation of the first rib require assistance from others for correction of the first rib, which is inconvenient and often results in poor correction outcomes.

### SUMMARY

The present disclosure aims to solve at least one of the technical problems in the existing technologies. In view of this, the present disclosure proposes a correction device which is easy for patients to use and improves the correction effect of the correction device.

According to an embodiment of the present disclosure, there is provided a correction device for correcting a first rib. The correction device includes a housing assembly, an electromagnet, a correction assembly, a mounting assembly and a control assembly. The housing assembly is provided with a receiving cavity, the receiving cavity having an clearance hole; the electromagnet includes a coil and an armature, the coil having an clearance channel, and the armature passing through the clearance channel and being movable in an axial direction of the coil; the correction assembly is disposed on the housing assembly and movable along the axial direction of the coil, an end of the correction assembly being connected to an end of the armature, and a further end of the correction assembly passing through the clearance hole; the control assembly is disposed on the housing assembly and is electrically connected to the coil to control the coil to be energized or de-energized; and the mounting assembly is disposed on the housing assembly, the mounting assembly being configured to mount the correction device to a corresponding support, where the armature, upon the coil being energized, pushes the further end of the correction assembly to move in a direction extending out of the housing assembly.

The correction device according to the embodiment of the present disclosure has at least the following beneficial effects.

When using the correction device, a patient can install the housing assembly onto a support via the mounting assembly. Then, the patient abuts their clavicle against the further end of the correction assembly and activate the control assembly to energize the coil, so that the coil generates a magnetic force, causing the armature to move in the direction of exiting the clearance channel. The armature abuts against an end of the correction assembly, allowing the armature to push the correction assembly to extend out of the clearance hole, thereby displacing a partially dislocated first rib to correct the first rib. The correction device is convenient for the patient to control by themself, allowing for correction of their first rib without the help of others, is convenient to use, and provides a strong corrective effect.

According to some embodiments of the present disclosure, the correction assembly includes a correction member and a first piston, the first piston being connected to an end of the armature, the correction member passing through the clearance hole, and the first piston, upon the coil being energized, being able to push the correction member to extend out of the housing assembly.

According to some embodiments of the present disclosure, the correction assembly further includes a second piston, an end of the second piston being connected to the correction member, and upon the coil being energized, an end of the first piston away from the armature being able to abut against an end of the second piston away from the correction member.

According to some embodiments of the present disclosure, the correction assembly further includes a resetting member, the resetting member being disposed between the first piston and the second piston, and the resetting member being configured to reset the first piston.

According to some embodiments of the present disclosure, the correction assembly further includes an elastic member, the elastic member being configured to move the second piston in the direction extending out of the housing assembly.

According to some embodiments of the present disclosure, a guide structure is provided between the first piston and the second piston.

According to some embodiments of the present disclosure, the guide structure includes a guide shaft provided in the second piston and a guide hole provided in the first piston, the guide shaft passing through the guide hole and being connectable to a side wall of the guide hole.

According to some embodiments of the present disclosure, a limiting structure is provided between the second piston and the housing assembly, and the limiting structure is configured to limit a movement stroke of the second piston.

According to some embodiments of the present disclosure, the limiting structure includes a limiting recess provided in a side wall of the receiving cavity and a limiting projection provided on the second piston, the limiting projection being accommodated in the limiting recess and being able to abut against a side wall of the limiting recess.

According to some embodiments of the present disclosure, the mounting assembly includes a first clamping member and a second clamping member arranged opposite each other, the housing assembly is provided with a first connecting portion and a second connecting portion arranged opposite each other, the first clamping member is disposed on the first connecting portion, the second clamping member is disposed on the second connecting portion, the first clamping member is configured to abut against a side of the support, and the second clamping member is configured to abut against a further opposite side of the support.

Additional aspects and advantages of the present disclosure will be given in part in the following description, and will become apparent in part from the following description or be learned by practice of the present disclosure.

### BRIEF DESCRIPTION OF DRAWINGS

The above and/or additional aspects and advantages of the present disclosure will become apparent and readily understood from the description of embodiments made in conjunction with the following accompanying drawings, in which:

FIG. 1 is a schematic diagram of a correction device according to an embodiment of the present disclosure;

FIG. 2 is a top view of a correction device according to an embodiment of the present disclosure;

FIG. 3 is a cross-sectional view of a correction device taken along line A-A in FIG. 2;

FIG. 4 is an exploded view of a correction device according to an embodiment of the present disclosure;

FIG. 5 is an exploded view of a first piston and an armature according to an embodiment of the present disclosure; and

FIG. 6 is a schematic diagram of the assembly of a second piston and a guide shaft according to an embodiment of the present disclosure.

### LIST OF REFERENCE SIGNS

housing assembly 100, first housing 101, second housing 102, first end cover 103, second end cover 104, mounting bracket 105, receiving cavity 110, clearance hole 120, limiting recess 130, first connecting portion 140, second connecting portion 150, electromagnet 200, coil 210, clearance channel 211, armature 220, correction assembly 300, correction member 310, first piston 320, guide hole 321, flange 322, second piston 330, limiting projection 331, resetting member 340, elastic member 350, guide shaft 360, mounting assembly 400, first clamping member 410, second clamping member 420, control assembly 500, first adjustment assembly 600, first adjustment member 610, first connection member 620, second adjustment assembly 700, second adjustment member 710, and second connection member 720.

### DETAILED DESCRIPTION

Hereinafter, embodiments of the present disclosure will be described in detail. Examples of the embodiments are illustrated in the accompanying drawings, where the same or like reference numerals throughout the figures indicates the same or like elements having the same or like functions. The embodiments described below with reference to the accompanying drawings are exemplary and are intended only to explain the present disclosure instead of being construed as limiting the present disclosure.

In the description of the present disclosure, it should be understood that, descriptions relating to orientation, for example, orientation or positional relationships indicated by “up”, “down”, “front”, “back”, “left”, “right”, etc. are based on the orientation or positional relationships shown in the accompanying drawings, and are to facilitate the description of the present disclosure and simplify the description only, rather than indicating or implying that the apparatus or element referred to must have a specific orientation or be constructed and operated in a specific orientation, and therefore cannot be construed as limiting the present disclosure.

In the description of the present disclosure, the meaning of “several” is one or a plurality; the meaning of “a plurality of” is two or more; “greater than”, “less than”, “more than”, etc. are to be construed as excluding a given figure; and “above”, “below”, “within”, etc. are to be construed as including a given figure. If “first” and “second”, etc. are referred to, it is only for the purpose of distinguishing technical features, and shall not be construed as indicating or implying relative importance or implying the number of the indicated technical features or implying the sequence of the indicated technical features.

In the description of the present disclosure, unless otherwise explicitly defined, the terms such as “arrange”, “install”, and “connect” should be construed in a broad sense, and those skilled in the art can determine the specific meanings of the above terms in the present disclosure in a rational way in conjunction with the specific contents of the technical schemes.

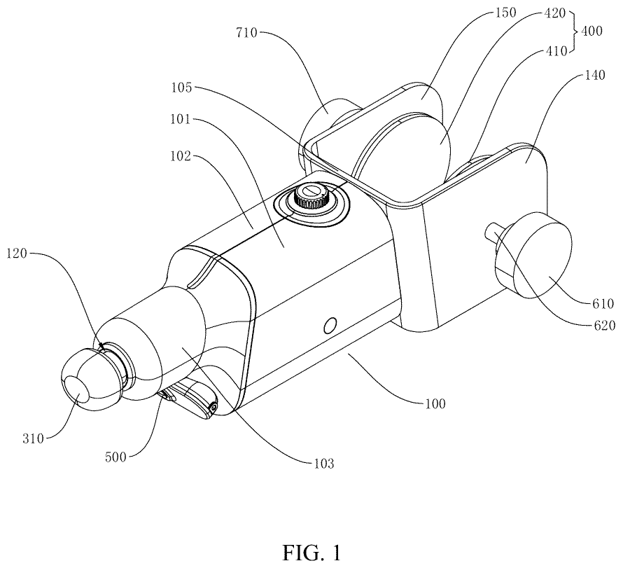

Referring to FIGS. 1 to 3, a correction device according to an embodiment the present disclosure includes a housing assembly 100, an electromagnet 200, a correction assembly 300, a mounting assembly 400 and a control assembly 500. The housing assembly 100 is provided with a receiving cavity 110, the receiving cavity 110 having a clearance hole 120. The electromagnet 200 includes a coil 210 and an armature 220, the coil 210 having a clearance channel 211, and the armature 220 passing through the clearance channel 211 and being movable in an axial direction of the coil 210. The correction assembly 300 is disposed on the housing assembly 100 and movable along the axial direction of the coil 210, an end of the correction assembly 300 being connected to an end of the armature 220, and a further end of the correction assembly 300 passing through the clearance hole 120. The control assembly 500 is disposed on the housing assembly 100 and is electrically connected to the coil 210 to control the coil 210 to be energized or de-energized. The mounting assembly 400 is disposed on the housing assembly 100 and is configured to mount the housing assembly 100 to a support. When the coil 210 is energized, the armature 220 pushes the further end of the correction assembly 300 to move in a direction extending out of the housing assembly 100, so that the further end of the correction assembly 300 can correct a first rib of a user. The correction device is convenient for a patient to control by themself, allowing for correction of their first rib without the help of others, is convenient to use, and provides a strong corrective effect.

Specifically, when using the correction device, a patient can install the housing assembly 100 onto a support via the mounting assembly 400. Then, the patient can abut their clavicle against the further end of the correction assembly 300 and activate the control assembly 500 to energize the coil 210, such that the coil 210 generates a magnetic force, causing the armature 220 to move in the direction of exiting the clearance channel 211. The armature 220 abuts against an end of the correction assembly 300, allowing the armature 220 to push the correction assembly 300 to extend out of the clearance hole 120, thereby displacing a partially dislocated first rib to correct the first rib. The correction device is convenient for a patient to control by themself, allowing for correction of their first rib without the help of others, is convenient to use, and provides a strong corrective effect.

It should be noted that the support may be a door body, a table or a column, and will not be described in detail here. When the support is a door body, the mounting assembly 400 clamps a free end of the door body, which can facilitate the mounting and fixation of the correction device for the convenience of the patient, and will not be described in detail herein. In addition, an end of the housing assembly 100, away from the correction assembly 300, is provided with a positioning surface. The positioning surface is configured to abut against a side of the door body, and the angle between the positioning surface and a moving plane of the correction assembly 300 is between 70 degrees and 80 degrees. In an embodiment, the angle between the positioning surface and the moving plane of the correction assembly 300 is 75 degrees. In other words, when the mounting assembly 400 is connected to the door body, the angle between the moving plane of the correction assembly 300 and a vertical direction is 15 degrees. This allows the correction assembly 300 to incline upward against the clavicle to correct the first rib, thereby improving the correction effect of the correction device.

It should be noted that when the coil 210 is energized, the correction assembly 300 impacts the clavicle of the patient with a force in the range of 20 to 30 pound-forces from within the housing assembly 100. The patient can choose an impact force of 20, 25, or 30 pound-forces based on their condition, which is not limited herein.

It should be noted that the electromagnet 200 further includes a capacitor, and the control assembly 500 is electrically connected to the coil 210 through the capacitor. When the electromagnet 200 is activated, the capacitor is charged and then discharged to store electricity for the coil 210, which will not be described in detail herein.

It can be understood that the control assembly 500 may be a switch structure, and the user can trigger the control assembly 500 by pressing or rotating, which will not be described in detail herein.

It should be noted that an induction switch is provided in the housing assembly 100, and the induction switch is electrically connected to the control assembly 500. When the patient abuts their clavicle with the other end of the correction assembly 300, the correction assembly 300 can trigger the induction switch, and the induction switch can trigger the control assembly 500 to cause the coil 210 to be energized, which will not be described in detail herein.

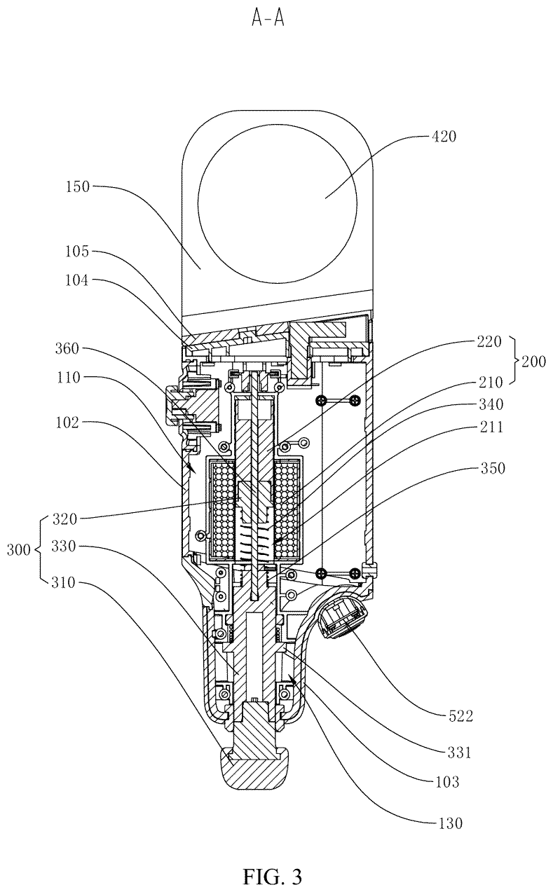

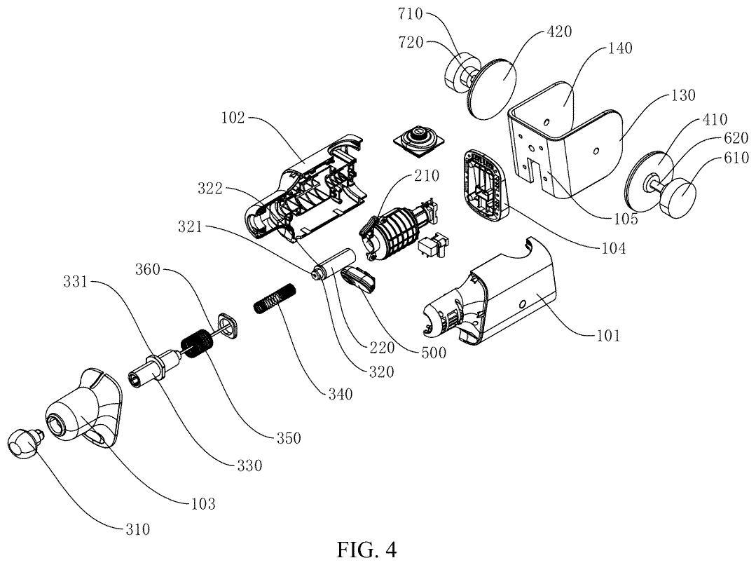

Referring to FIGS. 3 and 4, in some embodiments of the present disclosure, the correction assembly 300 includes a correction member 310 and a first piston 320. The first piston 320 is connected to an end of the armature 220. The correction member 310 passes through the clearance hole 120. The first piston 320, when the coil 210 is energized, is able to push the correction member 310 to move forward from the housing assembly 100, which can facilitate the correction of the first rib.

In an embodiment, the first piston 320 is fixedly connected to an end of the armature 220, and the correction member 310 passes through the clearance hole 120. When the coil 210 is energized, the first piston 320 can move in a direction approaching the correction member 310 to push the correction member 310 to move in a direction extending out of the housing assembly 100, which can facilitate correction of the first rib.

Referring to FIGS. 3 and 4, in some embodiments of the present disclosure, the correction assembly 300 further includes a second piston 330. An end of the second piston 330 is connected to the correction member 310. When the coil 210 is energized, an end of the first piston 320 that is away from the armature 220 can abut against an end of the second piston 330 that is away from the correction member 310.

In an embodiment, an end of the second piston 330 away from the first piston 320 passes through the clearance hole 120 and can move axially along an axial direction of the coil 210, and the correction member 310 is located on the outside of the housing, with the first piston 320 and the second piston 330 spaced apart along the axial direction of the coil 210. When the coil 210 is energized, the armature 220 drives the first piston 320 to move in a direction approaching the second piston 330. When the first piston 320 abuts against the second piston 330, the armature 220 simultaneously drives the second piston 330 to move in the direction extending out of the housing assembly 100, allowing the correction member 310 to apply an impact force to the clavicle of the patient, which can facilitate the correction of the first rib.

It should be noted that the first piston 320 and the second piston 330 can have an integral structure or a split structure, as long as it is convenient for the armature 220 to push and move the correction member 310, which is not limited herein.

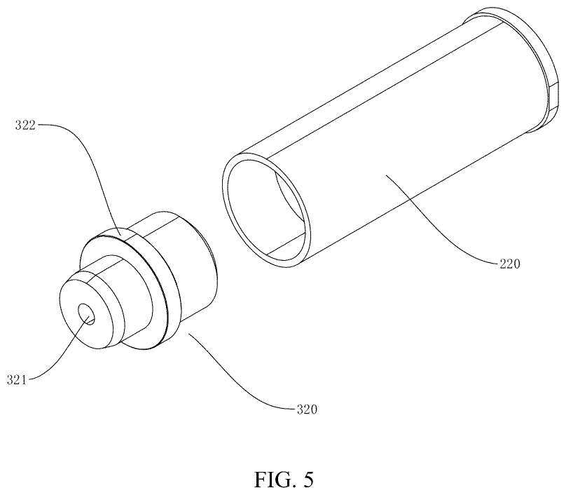

Referring to FIGS. 3 to 5, in some embodiments of the present disclosure, the correction assembly 300 further includes a resetting member 340. The resetting member 340 is disposed between the first piston 320 and the second piston 330, and the resetting member 340 is configured for resetting the first piston 320 such that the first piston 320 and the armature 220 can enter the clearance channel 211, so as to facilitate the coil 210 in driving the armature 220 again to move in the direction of exiting the clearance channel 211, which can enhance the user experience.

In an embodiment, the resetting member 340 is elastic, with an end abutting against an end of the first piston 320 and the other end abutting against an end of the second piston 330. When the coil 210 is de-energized, the magnetic force disappears, and the first piston 320 moves in the direction away from the correction member 310 under the action of the resetting member 340, allowing the first piston 320 and the armature 220 to enter the clearance channel 211. When the coil 210 is energized again, the coil 210 can drive the armature 220 to move in the direction of exiting the clearance channel 211, which can enhance the user experience.

It should be noted that the first piston 320 is provided with a flange 322, the flange 322 extends along a circumferential direction of the first piston 320, the second piston 330 is provided with a limiting projection 331, the limiting projection 331 extends along a circumferential direction of the second piston 330, and both ends of the resetting member 340 respectively abut against the flange 322 and the limiting projection 331, which will not be described in detail herein.

It should be noted that the resetting member 340 is a structure with axial elastic deformation, such as a compression spring or a rubber component, which is not limited herein.

Referring to FIGS. 3 and 4, in some embodiments of the present disclosure, the correction assembly 300 further includes an elastic member 350 for moving the second piston 330 in the direction extending out of the housing assembly 100, and when the patient completes a correction operation, the second piston 330 can automatically extend out of the clearance hole 120 to facilitate subsequent use by the patient.

In an embodiment, an end of the elastic member 350 abuts against the second piston 330, while the other end of the elastic member 350 abuts against the housing assembly 100. When the first piston 320 pushes the second piston 330 to move in the direction of extending out of the housing assembly 100, the elastic member 350 is compressed, so that the elastic member 350 obtains elastic potential energy. After the patient completes the correction operation, the coil 210 is de-energized, and the first piston 320 and the second piston 330 separate. The second piston 330 then moves in the direction of entering the housing assembly 100 under the action of the elastic member 350, allowing the second piston 330 to automatically reset for subsequent use by the patient.

Referring to FIGS. 3, 5 and 6, in some embodiments of the present disclosure, a guide structure is provided between the first piston 320 and the second piston 330, and can guide the first piston 320 such that the first piston 320 can accurately abut against the second piston 330, thereby improving the reliability of use of the correction device.

Referring to FIGS. 3, 5 and 6, in some embodiments of the present disclosure, the guide structure includes a guide shaft 360 provided in the second piston 330 and a guide hole 321 defined in the first piston 320, the guide shaft 360 passes through the guide hole 321 and can be connected to a side wall of the guide hole 321, and can guide the first piston 320 such that the first piston 320 can accurately abut against the second piston 330, thereby improving the reliability of use of the correction device.

In an embodiment, an end of the guide shaft 360 is fixedly connected to the second piston 330, the guide shaft 360 passes through the guide hole 321, and the diameter of the guide shaft 360 matches the diameter of the guide hole 321, such that the guide shaft 360 can guide the first piston 320 and the first piston 320 can accurately abut against the second piston 330, thereby improving the reliability of use of the correction device.

It should be noted that the positions of the guide shaft 360 and the guide hole 321 may be interchangeable, that is, the guide shaft 360 is provided in the first piston 320 and the guide hole 321 is defined in the second piston 330, and such an arrangement can also provide guidance for the first piston 320, which is not limited herein.

In some particular embodiments, the guide structure may also be a structure in which a guide strip cooperates with a guide groove, which will not be described in detail herein.

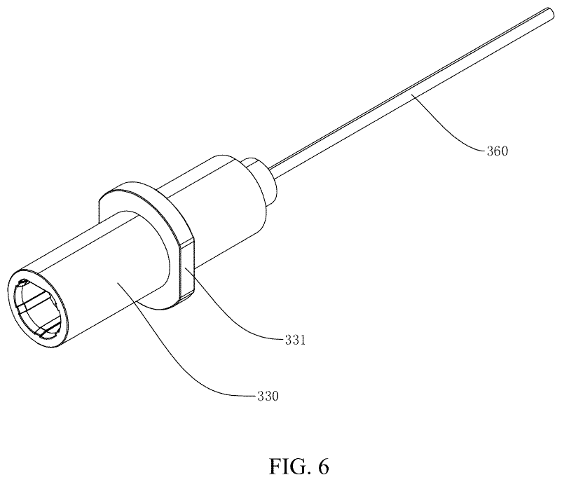

Referring to FIGS. 3 and 6, in some embodiments of the present disclosure, a limiting structure is provided between the second piston 330 and the housing assembly 100, and the limiting structure is configured to limit the movement stroke of the second piston 330, which can prevent situations where the movement stroke of the second piston 330 is too large so that the clavicle is impacted and injured by the correction member 310, and can improve the reliability of use of the correction device.

It should be noted that the movement stroke of the correction assembly 300 is between 5/16 inch and 7/16 inch, which is generally sufficient to achieve the desired displacement of the first rib and is not limited herein.

Referring to FIGS. 3 and 6, in some embodiments of the present disclosure, the limiting structure includes a limiting recess 130 defined in a side wall of the receiving cavity 110 and a limit protrusion 331 provided on the second piston 330. The limit protrusion 331 is accommodated in the limiting recess 130 and can abut against the side wall of the limiting recess 130, which can prevent situations where the movement stroke of the second piston 330 is too large so that the clavicle is impacted and injured by the correction member 310, and can improve the reliability of use of the correction device.

In an embodiment, the limiting recess 130 extends along an axial direction of the second piston 330. When the patient pushes the correction member 310 to move in a direction approaching the first piston 320, a side of the limiting projection 331 in the axial direction of the second piston 330 comes into contact with a side of the limiting recess 130. When the first piston 320 pushes the second piston 330 to its maximum stroke, the opposite side of the limiting projection 331 in the axial direction of the second piston 330 contacts the opposite side of the limiting recess 130, which can prevent situations where the movement stroke of the second piston 330 is too large so that the clavicle is impacted and injured by the correction member 310, and can improve the reliability of use of the correction device.

It should be noted that the limiting structure may also be a structure in which a limit pin and a limit hole cooperate with each other, the limit hole is provided on the side wall of the receiving cavity 110, the limit hole is of an oblong structure, the limit pin is provided on the second piston 330, and the limit pin passes through the limit hole. This arrangement can also limit the movement stroke of the second piston 330, which will not be described in detail herein.

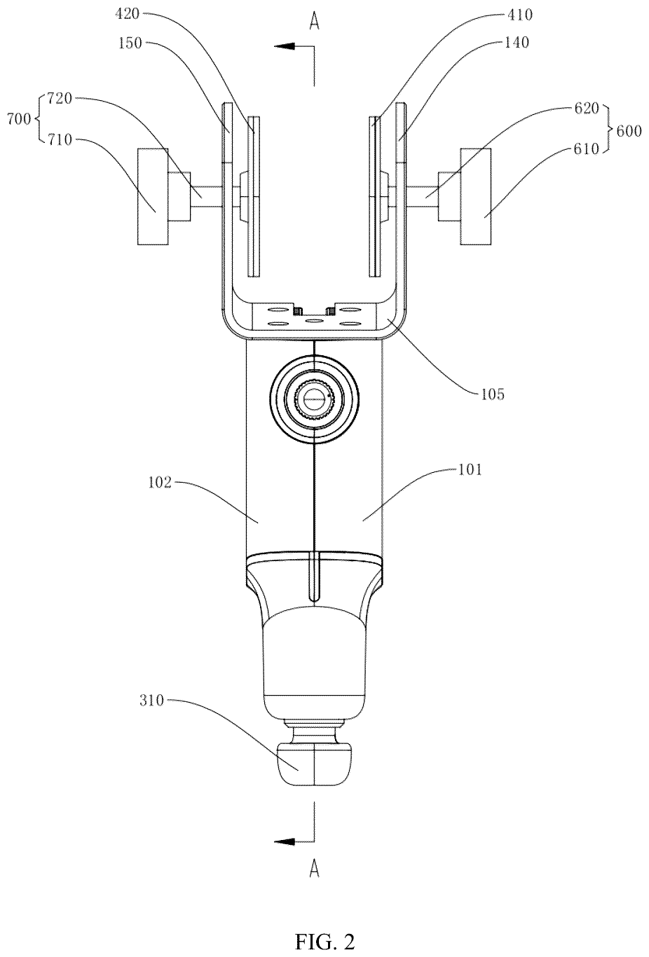

Referring to FIGS. 1 and 2, in some embodiments of the present disclosure, the mounting assembly 400 includes a first clamping member 410 and a second clamping member 420 arranged opposite each other. The housing assembly 100 is provided with a first connecting portion 140 and a second connecting portion 150 arranged opposite each other. The first clamping member 410 is disposed on the first connecting portion 140, the second clamping member 420 is disposed on the second connecting portion 150, the first clamping member 410 is configured to abut against a side of the support, and the second clamping member 420 is configured to abut against a further opposite side of the support, which can facilitate the mounting of the correction device.

In an embodiment, the housing assembly 100 is of a cylindrical structure, the clearance hole 120 is provided at an end of the housing assembly 100, the first connecting portion 140 and the second connecting portion 150 are provided opposite each other in a width direction of the housing assembly 100. When the correction device is in use, the first clamping member 410 is configured to abut against a side of the support, and the second clamping member 420 is configured to abut against a further opposite side of the support, which can facilitate the mounting of the correction device.

It should be noted that the mounting assembly 400 can alternatively be three clamping jaws, which can clamp a cylindrical support, which can also facilitate the mounting of the correction device, and will not be described in detail herein.

Referring to FIGS. 1 and 4, in some embodiments of the present disclosure, the first clamping member 410 and the housing assembly 100 are provided with a first adjustment assembly 600, and the first adjustment assembly 600 is configured for adjusting a distance between the first clamping member 410 and the second clamping member 420, to allow for adaptation to supports of different sizes, and can improve the versatility of the correction device.

In some embodiments of the present disclosure, the first adjustment assembly 600 includes a first adjustment member 610 and a first connection member 620. An end of the first connection member 620 is connected to the first clamping member 410, and the other end of the first connection member 620 is connected to the first adjustment member 610. The first connecting portion 140 is provided with a first threaded hole, the first connection member 620 is provided with a first threaded part, and the first threaded part is threadedly mated with the first threaded hole, to allow for adaptation to supports of different sizes, and can improve the versatility of the correction device.

In an embodiment, the first threaded portion is of a male threaded structure, while the first threaded hole is of a female threaded structure. The male threaded structure and the female threaded structure are threadedly mated, so that the user can adjust the first clamping member 410 to move in a direction approaching or away from the second clamping member 420 by rotating the first adjustment member 610. When the support is a door body, the mounting assembly 400 can adapt to the door body of different thicknesses, and can facilitate the mounting of the correction device, so as to improve the versatility of the correction device.

It should be noted that the first adjustment assembly 600 can alternatively be a rack-and-pinion structure or an eccentric wheel locking structure, which will not be described in detail herein.

It should be noted that the first adjustment member 610 and the first connection member 620 are an integral structure, which can reduce the number of molds, reduce the production and manufacturing costs of molds, and thus reduce the production and manufacturing costs of the correction device, and will not be described in detail herein.

Referring to FIGS. 1 and 4, in some embodiments of the present disclosure, the second clamping member 420 and the housing assembly 100 are provided with a second adjustment assembly 700, and the second adjustment assembly 700 is configured for adjusting a distance between the first clamping member 410 and the second clamping member 420, to allow for adaptation to supports of different sizes, and can improve the versatility of the correction device.

In an embodiment, the second adjustment assembly 700 includes a second adjustment member 710 and a second connection member 720. An end of the second connection member 720 is connected to the second clamping member 420, and the other end of the second connection member 720 is connected to the second adjustment member 710. The second connecting portion 150 is provided with a second threaded hole, the second connection member 720 is provided with a second threaded part, and the second threaded part is threadedly mated with the second threaded hole, to allow for adaptation to supports of different sizes, and can improve the versatility of the correction device.

In some embodiments of the present disclosure, the first connecting portion 140 and the second connecting portion 150 are provided on a side of the housing assembly 100 away from the clearance hole 120, and when the correction device is mounted, the opposite end of the housing assembly 100 (the end away from the clearance hole 120) can abut against the support, so that the support can provide an acting force opposite to the impact force of the correction member 310, and the correction device can be stably fixed.

Referring to FIG. 4, in some embodiments of the present disclosure, the housing assembly 100 includes a first housing 101, a second housing 102, a first end cover 103, a second end cover 104 and a mounting bracket 105. The first housing 101, the second housing 102, the first end cover 103 and the second end cover 104 enclose to form the receiving cavity 110. The mounting bracket 105 is connected to the first housing 101 and/or the second housing 102, with the first connecting portion 140 and the second connecting portion 150 both disposed on the mounting bracket 105. The clearance hole 120 is defined in the first end cover 103. The mounting bracket 105 is connected to the second end cover 104 by means of bolts or snap-fit, and the first housing 101 and second housing 102 are connected by snap-fit or bolts, facilitating the assembly of the correction device.

The various technical features of the embodiments described above can be arbitrarily combined. For the sake of simplicity of description, not all possible combinations of the various technical features of the above embodiments are described; however, combinations of these technical features should be deemed as being within the scope of this specification only if there exists no contradiction therebetween.

Although the embodiments of the present disclosure have been described in detail above with reference to the accompanying drawings, the present disclosure is not limited to the above embodiments, and various changes may be made within the knowledge of those of ordinary skill in the art without departing from the purpose of the present disclosure.

What is claimed is:

1. A correction device for correcting a first rib, comprising:
a housing assembly provided with a receiving cavity, the receiving cavity having a clearance hole;
an electromagnet comprising a coil and an armature, the coil having a clearance channel, and the armature passing through the clearance channel and being movable in an axial direction of the coil;
a correction assembly disposed on the housing assembly and movable along the axial direction of the coil, an end of the correction assembly being connected to the armature, and a further end of the correction assembly passing through the clearance hole;
a control assembly disposed on the housing assembly, the control assembly being electrically connected to the coil to control the coil to be energized or de-energized; and
a mounting assembly disposed on the housing assembly, the mounting assembly being configured to mount the correction device to a support,
wherein the armature is configured to, upon the coil being energized, push the further end of the correction assembly to move in a direction extending out of the housing assembly.

2. The correction device of claim 1, wherein the correction assembly comprises a correction member and a first piston, the first piston being connected to an end of the armature, the correction member passing through the clearance hole, and the first piston, upon the coil being energized, being able to push the correction member to extend out of the housing assembly.

3. The correction device of claim 2, wherein the correction assembly further comprises a second piston, an end of the second piston being connected to the correction member, and the first piston is configured in such a way that, upon the coil being energized, an end of the first piston away from the armature abuts against an end of the second piston away from the correction member.

4. The correction device of claim 3, wherein the correction assembly further comprises a resetting member, the resetting member being disposed between the first piston and the second piston, and the resetting member being configured to reset the first piston.

5. The correction device of claim 2, wherein the correction assembly further comprises an elastic member, the elastic member being configured to move the second piston in the direction extending out of the housing assembly.

6. The correction device of claim 3, wherein a guide structure is provided between the first piston and the second piston.

7. The correction device of claim 6, wherein the guide structure comprises a guide shaft provided in the second piston and a guide hole provided in the first piston, the guide shaft passing through the guide hole and being connectable to a side wall of the guide hole.

8. The correction device of claim 6, wherein a limiting structure is provided between the second piston and the housing assembly, and the limiting structure is configured to limit a movement stroke of the second piston.

9. The correction device of claim 6, wherein the limiting structure comprises a limiting recess provided in a side wall of the receiving cavity and a limiting projection provided on the second piston, the limiting projection being accommodated in the limiting recess and being able to abut against a side wall of the limiting recess.

10. The correction device of claim 1, wherein the mounting assembly comprises a first clamping member and a second clamping member arranged opposite each other, the housing assembly is provided with a first connecting portion and a second connecting portion arranged opposite each other, the first clamping member is disposed on the first connecting portion, the second clamping member is disposed on the second connecting portion, the first clamping member is configured to abut against a side of the support, and the second clamping member is configured to abut against a further opposite side of the support.
